SEQUENCE LISTING (17 sequences)

SEQ ID NO: 1 - DNA, 450 bp, Nicotiana tobacum
  PR1b promoter sequence
aaattctttttccaatggacattcccattctgaaacaaaagagatataaatatcgaagcaaaattaatcagatcgttaaatgtagaaaatattagaagaattaacacagtaaccataaccaagtaaccataaccagtctattttatttaacaaaaaacacatctactagatcaaaaaagtgtttaacttcatgcatggacaatttaaaattattttgcaacatcaggtaaaatattttacaataattggtaattgcttataagtttaaaatatgataacctagaaaataggataaataactatctataacaagatatattacattgatattaccatgtcaaaaatttttagtaagtgcatgaatgatcgccgtgaaatcttcaagatttctcctataaatacctgtggtagtaaatctagtttttccattcaaaataaaacatttctcct

SEQ ID NO: 2 - DNA, 29 bp, Artificial Sequence
  Description of Artificial Sequence Synthetic PR1b 5′ primer
tttgaattcaaattctttttccaatggac

SEQ ID NO: 3 - DNA, 29 bp, Artificial Sequence
  Description of Artificial Sequence Synthetic PR1b 3′ primer
ttaggatccgagaaatcttttattttgaa

SEQ ID NO: 4 - DNA, 528 bp, Artificial Sequence
  Description of Artificial Sequence Octopine- type T-DNA, carried by the Ti plasmid pTiAch5
ggatccatcaaatcttccagctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttcccgccggcgcgcatgggtgagattccttgaagttgagtattggctgcag

SEQ ID NO: 5 - DNA, 29 bp, Artificial Sequence
  Description of Artificial Sequence Synthetic OCS 5′ primer
tttggatccatcaaatcttccagctgctt

SEQ ID NO: 6 - DNA, 29 bp, Artificial Sequence
  Description of Artificial Sequence Synthetic OCS 3′ primer
tttctgcagccaatactcaacttcaagga

SEQ ID NO: 7 - DNA, 23 bp, Artificial Sequence
  Description of Artificial Sequence pUC119 XhoI-XbaI adapter oligonucleotide
agctctcgagagatctagacatg

SEQ ID NO: 8 - DNA, 15 bp, Artificial Sequence
  Description of Artificial Sequence pUC119 XhoI-XbaI adapter oligonucleotide
tctagatctctcgag

SEQ ID NO: 9 - DNA, 763 bp, Artificial Sequence
  Description of Artificial Sequence Synthetic CaMV S35 promoter sequence
ctgcagacaagaagaaaatcttcgtcaacatggtggagcacgacacgcttgtctacctccaaaaatatcaaagatacagtctcagaagaccaaagggaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacgcttgtctacctccaaaaatatcaaagatacagtctcagaagaccaaagggaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagcttaaagtcgac

SEQ ID NO: 10 - DNA, 29 bp, Artificial Sequence
  Description of Artificial Sequence Synthetic CaMV S35 5′ promoter sequence
tttctgcagacaagaagaaaatcttcgtc

SEQ ID NO: 11 - DNA, 38 bp, Artificial Sequence
  Description of Artificial Sequence Synthetic CaMV 3′ promoter sequence
tttgtcgactttaagcttccttatatagaggaagggtc

SEQ ID NO: 12 - DNA, 1095 bp, Escherichia coli
  lac operator sequence
agatctatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaagatct

SEQ ID NO: 13 - DNA, 22 bp, Artificial Sequence
  Description of Artificial Sequence Synthetic HinDIII oligonucleotide
agctattgtgagcgctcacaat

SEQ ID NO: 14 - DNA, 22 bp, Artificial Sequence
  Description of Artificial Sequence Synthetic SalI oligonucleotide
tcgaattgtgagcgctcacaat

SEQ ID NO: 15 - DNA, 11 bp, Artificial Sequence
  Description of Artificial Sequence Synthetic SacI adaptor oligonucleotide
tcgagagctct

SEQ ID NO: 16 - DNA, 11 bp, Artificial Sequence
  Description of Artificial Sequence Synthetic SacI adaptor oligonucleotide
ctagagagctc

SEQ ID NO: 17 - DNA, 1746 bp, Saccharomyces cerevisiae
  Yeast PAB1 gene
ctcgagatggctgatattactgataagacagctgaacaattggaaaacttgaatattcaagatgaccaaaagcaagccgccactggttcagaaagccaatctgttgaaaactcttctgcatcattatatgttggtgacttagaaccttctgtttccgaagcccacttatatgatatcttctctccaatcggttcagtctcctccattcgtgtctgtcgtgatgccatcactaagacctctttgggctatgcttatgttaactttaacgaccatgaagccggcagaaaagcaattgagcaattgaactacactccaatcaagggtagattatgccgtattatgtggtctcaacgtgacccatcattgagaaagaagggttctggtaacatctttatcaagaacttgcaccctgatattgacaacaaggctttgtatgacactttctctgtgtttggtgacatcttgtccagcaagattgccaccgacgaaaacggaaaatccaagggttttgggtttgttcacttcgaagaagaaggtgctgccaaggaagctattgatgctttgaatggtatgctgttgaacggtcaagaaatttatgttgctcctcacttgtccagaaaggaacgtgactctcaattggaagagactaaggcacattacactaacctttatgtgaaaaacatcaactccgaaactactgacgaacaattccaagaattgtttgccaaatttggtccaattgtttctgcctctttggaaaaggatgctgatggaaaattgaagggtttcgggtttgttaactacgaaaagcatgaagacgctgtgaaagctgttgaagctttgaatgactctgaactaaatggagaaaagttatacgttggtcgtgcccaaaagaagaatgaacgtatgcatgtcttgaagaagcaatacgaagcttacagattggaaaaaatggccaagtaccaaggtgttaatttgtttgttaagaacttagatgacagcgttgatgacgaaaagttggaagaagaatttgctccatatggtactatcacttctgcaaaggttatgagaaccgaaaacggtaagtctaagggttttggttttgtttgtttctcaactccagaggaagctactaaggccattacagaaaagaaccaacaaattgttgctggtaagccattatacgttgccattgctcaaagaaaagacgtaagacgttctcaattggctcaacaaatccaagccagaaatcaaatgagataccagcaagctactgctgccgctgccgccgccgctgccggtatgccaggtcaattcatgcctccaatgttctatggtgttatgccaccaagaggtgttccattcaacggtccaaacccacaacaaatgaacccaatgggcggtatgccaaagaacggcatgccacctcaatttagaaatggtccggtttacggcgtccccccacaaggtggtttcccaagaaatgccaacgataacaaccaattttatcaacaaaagcaaagacaagctttgggtgaacaattatacaagaaggtttctgctaagacttcaaatgaagaagcagctggtaaaattactggtatgattttggatttgccacctcaagaggtcttcccattgttggaaagtgatgaattgttcgaacaacactacaaagaagcttctgctgcctatgagtctttcaaaaaggagcaagaacaacaaactgagcaagcttaagagctc

(19) United States
(12) Patent Application Publication
(10) Pub. No.: US 2003/0040102 A1
(43) Pub. Date: Feb. 27, 2003

(54) Feedback-regulated expression system and uses thereof

(76) Inventors: Arthur G. Hunt, Lexington, KY (US); Qingshun Li, Mason, OH (US); Tomal Dattaroy, Chennai (IN)

Correspondence Address: MCDERMOTT WILL & EMERY, 600 13TH STREET, N.W., WASHINGTON, DC, 20005-3096, US

(21) Appl. No.: 10/162,214

(22) Filed: Jun. 5, 2002

Related U.S. Application Data
(60) Provisional application No. 60/295,565, filed on Jun. 5, 2001.

(51) Int. Cl. A01H 1/00; C07H 21/04; C12N 1/21; C12N 15/87

(52) U.S. Cl. 435/252.1; 800/294; 800/288; 435/320.1; 536/23.2; 435/469

(57) ABSTRACT

A feedback-regulated expression system comprising a nucleic acid construct comprising a first polynucleotide encoding an elicitin operably linked to a first plant promoter comprising at least one E. coli lac operator (LacO) located between the promoter TATA box and the translation initiation site of the first polynucleotide, wherein the first plant promoter is constitutive; and a second polynucleotide encoding an E. coli lac repressor (LacI) operably linked to a PR gene is described. the feedback-regulated expression system is used to generate transgenic plants that have enhanced resistance to plant pathogens.

This application claims priority to application 60/295,565, filed Jun. 5, 2001.

### FIELD OF THE INVENTION

The present invention relates to gene constructs and methods for regulating the expression of genes in plants. More particularly, the invention relates to gene constructs for feedback regulated expression of plant genes to maintain systemic acquired resistance in plants.

### BACKGROUND OF THE INVENTION

Plants possess the capability to acquire an enhanced level of broad-spectrum resistance following a primary infection, usually involving a necrotizing pathogen. This phenomenon is commonly referred to as systemic acquired resistance (SAR) and has been shown to occur in both dicotyledenous as well as monocotolydenous plants (Ryals et al., 1996; Sticher et al., 1997; Morris et al., 1998). Establishment of SAR is accompanied by a local and systemic increase in salicylic acid (SA) and the expression of a subset of PR genes encoding extracellular proteins.

The properties of plants that are induced for SAR are attractive from the perspective of pathogen resistance: they are usually protected against a broad range of bacterial, fungal, and viral pathogens, yet they may display little or no harmful effects otherwise (e.g., serious yield losses, aberrant developmental patterns, etc.). Consequently, it is of great interest to explore strategies whereby constitutive or tightly controlled induction of SAR is achieved.

SAR can be induced by several factors. For example, challenge with so-called incompatible pathogens, which necessarily leads to a hypersensitive response, induces SAR (Sticher et al., 1997). Challenge with non-pathogenic microbes can also induce SAR (van Loon et al., 1998). Certain chemicals may be able to induce SAR in treated plants (Gorlach et al., 1996; Morris et al., 1998; Rao et al., 1999). The expression of any of a number of genes that, while not of pathogenic origin per se, can induce hypersensitive responses or cause disease-like lesions, and can trigger SAR, apparently through a means similar to that by which incompatible pathogens induce SAR (Dangl et al., 1996).

In light of the range of stimuli known to induce SAR, several strategies have been tested to genetically engineer plants so that they are constitutive for SAR, or can be induced with agents not usually associated with disease and defense responses. Expression of both plant resistance and microbial avr genes in the same plant has been tested; when the avr gene is controlled by a promoter whose activity is induced upon challenge by pathogens (including those unrelated to the source of the avr gene), the resulting plants can respond to so-called compatible pathogens as if possessing a specific gene-for-gene system (Hammond-Kosack et al., 1994, 1998). Constitutive expression of genes whose products act downstream from the putative receptors can result in constitutive SAR (Oldroyd et al., 1998). Interestingly, in some instances, the resulting plants displayed few (if any) detrimental side effects, indicating that it is possible to condition permanent SAR without seriously affecting plant growth and development, or crop yield (Bowling et al., 1997; Yu et al., 1998; Oldroyd et al., 1998).

Induced or constitutive expression of microbial avr genes, elicitor or elicitor-like genes and other so-called disease lesion-mimic genes can also induce SAR constitutively in plants (Dangl et al., 1996). In these instances, constitutive induction of SAR is accompanied by a diminished growth habit, and by the expected consequences of the expression of genes associated with cell death. This is because the products of most genes used for this purpose are themselves inherently toxic to cells, and because most expression regimes, even those purported to yield regulated expression, are “leaky” enough to result in low levels of production of highly toxic gene products.

There is a long felt need in the art for methods of protecting plants, particularly crop plants, from infection by plant pathogens, including phytopathogenic viruses, fungi and bacteria. There is also a long felt need in the art for plant transcriptional regulatory sequences for use in controlling the SAR response in transgenic plants. Thus, there is a need for a system that can induce SAR in plants so as to provide the benefits of the SAR response without unwanted side effects associated with constitutive SAR expression.

### BRIEF DESCRIPTION OF THE DRAWINGS

FIG. 1 is an illustration of the micro-T-DNA of the invention that carries the SAR-regulated lacI gene and lacI-regulated PAB1 gene.

FIG. 2 is a Northern blot analysis of R0 plants that carry the construct shown in FIG. 1. Each panel represents the results of one gel; negative (−) and positive (+) controls are indicated. Positive controls are untransformed plants that have been treated with salicylic acid, a treatment known to induce PR1a gene expression. Negative controls are from untransformed plants grown under the same conditions as the various transformed plants analyzed in this study.

FIG. 3 demonstrates the resistance of CC lines to Erwinia caratovora. The seed of BY21 WT and transgenic lines CC-2 (#11, 15, 26, 27, 30, 38, 40, and 48) were tested.

FIG. 4 demonstrates the resistance of CC lines to Pseudomonas syringae.

### DETAILED DESCRIPTION OF THE INVENTION

The present inventors have established that foreign genes that can act as lesion-mimics or whose constitutive expression can induce cell death, associated hypersensitive responses and/or SAR in plants can be used in a novel feedback-regulated expression system to effect broad-range protection of plants from pathogens. The feedback-regulated expression system of the invention links the expression of genes whose products are cytotoxic to plants to the establishment of systemic acquired resistance. The cytotoxic gene product is an elicitin, which is a substance, usually a polypeptide, that induces localized cell death or a hypersensitive response in plants. Generally, constitutive expression of an elicitin in plants is usually accompanied by modest to severe impairment of growth. Elicitins are produced by plant pathogens and potential plant pathogens, which induce a hypersensitive response in plants. The amino acid and coding sequences of many eliciting, including the plant-pathogenic gene, ParA1 of Phytophthora parasitica, an avr gene of Fulva fulvia, an avr gene of Cladosporium fulva, multiple homologs of the avirulence gene, avrBs3, of Xanthomonas, the avrD gene of Pseudomonas syringae pv. tomato, the avrD gene of P. syringae pv. glycinea, are well known.

It is understood that to be useful in the present invention as it applies to creating transgenic plants with improved disease resistance traits using an elicitin coding sequence expressed under the regulatory control of the feedback-regulation expression system that the elicitin must be able to induce activity of the defense gene promoter, which is a constituent of the feedback-regulation expression system (a PR promoter, including, but not limited to promoters of the genes governing phytoalexin synthesis, the hypersenitive response, and or localized necrosis) in the plants.

A transgenic plant is one which has been genetically modified to contain and express heterologous DNA sequences, either as regulatory RNA molecules or as proteins. As specifically exemplified herein, a transgenic plant is genetically modified to contain and express an elicitin encoding DNA sequence operably linked to and under the regulatory control of a feedback-regulation expression system. As used herein, a transgenic plant also refers to those progeny of the initial transgenic plant which carry and are capable of expressing the elicitin coding sequence under the regulatory control of the feedback-regulation expression system described herein. Seeds containing transgenic embryos are encompassed within this definition.

The feedback-regulated expression system of the invention comprises a first polynucleotide encoding an elicitin that is cytotoxic to plant cells operably linked to a first constitutive plant promoter comprising at least one E. coli lac operator (LacO) located between the promoter TATA box and the translation initiation site of the first polynucleotide and a second polynucleotide encoding an E. coli lac repressor (LacI) operably linked to a PR gene promoter. In a preferred embodiment, the feedback-regulated system comprises two E. coli lac operators aligned in tandem. The first plant promoter may be any constitutive plant promoter that can be modified to include an E. coli lac operator, e.g., a caulimovirus 35S promoter s (e.g., figwort mosaic virus), ACT2 from Arabidopsis, tCUP from tobacco and the like.

Preferably, the first plant promoter is a modified CaMV 35S promoter or a modified minimal promoter fragment of the CaMV 35S promoter that has been modified to contain at least one E. coli lac operator downstream of the promoter TATA box. The PR gene promoter preferably is the PR-1b promoter.

The elicitin encoded by the first polynucleotide of the feedback-regulation system of the invention can be any polypeptide or factor whose constitutive expression induces localized cytotoxic effects (cell death) in plant cells, or causes a severe to mild hypersenitive response, such as, for example, yeast PABP, Pseudomonas Syringae HrmA, an avrD, avr, chitinase, Beta-1,3-glucanase, thaumatin-like protein, or avrs3. In preferred embodiments of the invention, the polynucleotide encodes a yeast poly(A) binding protein (Pab1p) [Adam et al., 1986] or a Pseudomonas syringae pv. syringae HrmA elicitin gene [Alfano et al., 1997]. Each of the aforementioned genes can act as a lesion-mimic and as an inducer of SAR.

The studies presented herein establish that use of an elicitin gene in the feedback-regulated expression system of the invention protects transgenic plants in which their expression levels are below those that cause substantial cell death. This observation is important, as it indicates that regulated expression, even with somewhat “leaky” promoters, is a viable strategy for using these genes to effect broad-range protection of plants against pathogens.

The present invention provides a regulated expression regime for maintaining the SAR status of a plant. By linking the expression of a cytotoxic elicitin gene to the SAR status of the plant, the plant can be conditioned for constitutive SAR while avoiding the deleterious effects associated with constitutive expression of the cytotoxic gene. Plants that possess the feedback-regulated construct of the invention are resistant to different types of pathogens, thereby demonstrating the utility of the regulated strategy for the production of plants that are conditioned for resistance to a broad range of pathogens.

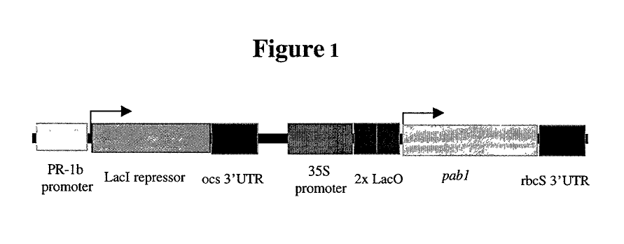

The basic strategy to extend the usefulness of plant cytotoxic genes or elicitins, such as the yeast PAB1 gene, for disease resistance in plants is to make the expression of such genes dependent on the establishment of systemic acquired resistance. Briefly, the approach used herein to develop the feedback-regulated expression system was to place the cytotoxic gene (e.g., the PAB1 or HrmA gene) gene under control of a modified constitutive promoter, such as the CaMV 35S promoter, wherein the modification entailed the introduction of at least one, and preferably two copies of an E. coli lac operator sequence (Brown et al., 1987) between the TATA box and the or the HrmA gene. This construct was combined with one in which the lac repressor (Brown et al., 1987) was controlled by a PR promoter. In a preferred embodiment the second promoter is the tobacco PR-1b gene promoter (FIG. 1), although any promoter that is inducible by the elicitin encoded by the first polynucleotide may be used.

The rationale behind this construct is as follows: if the cytotoxic gene (e.g., the PAB1 gene or HrmA gene) is expressed in plant cells, it induces both localized cell death and systemic resistance (Li et al., 2000). Moreover, in the case of the PAB1 gene, given the growth stage dependence of the effects of constitutively-expressed PAB1 (Li et al., 2000), it is likely that older tissues would probably be the first to experience the induction of localized cell death. Thus, in parts of older leaves (which may be imperceptible), the cytotoxic gene expression (for example, the PAB1 gene expression) should induce localized cell death, in turn eliciting SAR. The induction of SAR, in turn, is be accompanied by the activation of the PR promoter, followed by accumulation of the lac repressor. This, in turn, shuts off expression of the cytotoxic gene (e.g., the PAB1 gene) in tissues not yet affected by the expression of this gene, thereby preserving a relatively normal growth habit. The net result is morphologically normal plants that are conditioned for constitutive expression of SAR.

The effects of the feedback-regulated expression system of the invention have been tested on two different pathogens. The results indicate that this approach should be effective against any disease (of fungal or bacterial origin) that can be abrogated by the establishment and maintenance of systemic acquired resistance.

The construct shown in FIG. 1 was assembled and introduced into tobacco and Arabidopsis by Agrobacterium-mediated transformation. In contrast to what was reported for the transformation of plants with a constitutive PAB1 gene (Li et al., 2000), it was relatively easy to obtain plants with the feedback-regulated expression construct of the invention (the CC construct). Moreover, all of the plants that were obtained were normal in appearance and growth habit (not shown). In contrast, more than half of the primary transformants carrying a 35S-PAB1 construct were impaired in growth and development (Li et al., 2000). The presence of the PAB1 and lacI constructs in the various primary transformants was confirmed by PCR (not shown). A number of these plants were selected for further analysis, as described in the Examples.

The principal screen that was used to identify plants that might possess enhanced disease resistance characteristics was one for constitutive PR gene expression. The rationale flows from the strategy described above and—as long as PR gene expression is activated, the lac repressor is expressed, the PAB1 gene or other cytotoxic gene will be repressed, and plants will be free from the deleterious effects of PAB1 gene or other cytotoxic gene expression.

A transgenic plant of the invention can be produced by any means known to the art, including but not limited to Agrobacterium tumefaciens-mediated DNA transfer, preferably with a disarmed T-DNA vector, electroporation, direct DNA transfer, and particle bombardment. Techniques are well-known to the art for the introduction of DNA into monocots as well as dicots, as are the techniques for culturing such plant tissues and regenerating those tissues. Monocots which have been successfully transformed and regenerated include wheat, corn, rye, rice and asparagus. For efficient production of transgenic plants, it is desired that the plant tissue used for transformation possess a high capacity for regeneration.

Techniques for genetically engineering plant cells and/or tissue with an expression cassette comprising a recombinant nucleic acid include, for example, Agrobacterium-mediated transformation, electroporation, microinjection, particle bombardment or other techniques known to the art. The expression cassette may further contain a marker allowing selection of the heterologous DNA in the plant cell, e.g., a gene carrying resistance to an antibiotic such as kanamycin, hygromycin, gentamicin, or bleomycin.

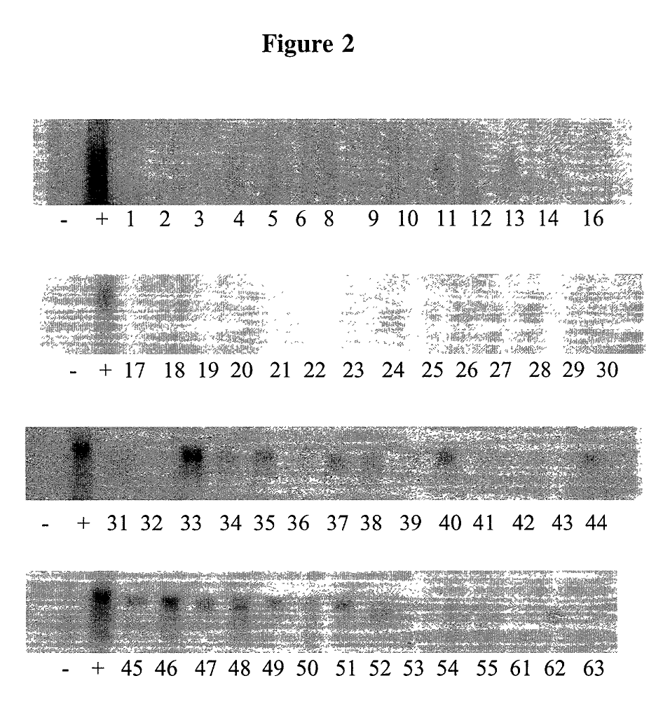

Taken together, the data obtained with transgenic plants containing a construct of the invention, and independent challenges of transgenic plants containing the constructs with different plant pathogens demonstrate that the feedback-regulated expression strategy illustrated in FIG. 1 is effective in conditioning transgenic plants for enhanced resistance to disease. This is presumably due to the constitutive establishment of a state of systemic acquired resistance, as indicated by the expression of the elicitin gene in the transformed cell lines (FIG. 2). This approach should be effective against any disease (of fungal or bacterial origin) that can be abrogated by the establishment and maintenance of systemic acquired resistance. The expression strategy should also be effective in conjunction with any foreign gene whose constitutive expression induces cell death and/or associated hypersensitive responses and SAR.

All references cited in the present application are expressly incorporated by reference thereto.

The following examples are provided for illustrative purposes and are not intended to limit the scope of the invention as claimed herein. Ant variations in the exemplified compositions and methods which occur to the skilled artisan are intended to fall within the scope of the present invention.

### EXAMPLE 1

The construct illustrated in FIG. 1 was assembled as follows. The tobacco PR1b promoter SEQ ID NO. 1) was amplified by PCR using tobacco genomic DNA as a template; EcoRI and BamHI sites were incorporated into the PCR primers so that an orientation 5′-EcoRI-PR1b promoter-BamHI-3′ was obtained. (PR1b primers: 5′-tttgaattcaaattctttttccaatggac (SEQ ID NO. 2) and 3′-ttaggatccgagaaatcttttattttgaa (SEQ ID NO. 3). The PCR product was digested with EcoRI and BamHI and cloned into pUC119 that had been digested with the same enzymes. The resulting clone—pUC119:PR1bP—was digested with BamHI and PstI and ligated with a PCR product containing the octopine synthase polyadenylation signal. For this, the ocs poly(A) signal (SEQ ID NO. 4) was amplified using a pBluescript clone (MacDonald et al., 1991), using primers with BamHI and PstI sites that were suited for this cloning (ocs primers: 5′-tttggatccatcaaatcttccagctgctt(SEQ ID NO. 5) and 3′-tttctgcagccaatactcaacttcaagga (SEQ ID NO. 6). The resulting plasmid, pUC119:PR1bP:ocs3′, was digested with BamHI and ligated with a BglII fragment from pMTLacI (Brown et al., 1987) containing the lacI gene. The ligations were treated with BamHI before transformation, and recombinants with the appropriate orientation of the lacI gene identified by restriction enzyme digestion. This plasmid was the digested with SphI and HindIII and the pUC119 XhoI-XbaI adapter oligonucleotides (A: agctctcgagagatctagacatg (SEQ ID NO. 7) and B: tctagatctctcgag (SEQ ID NO. 8) ligated to the digested DNA. This plasmid was termed pTD1.

In parallel, a duplicated CaMV 35S promoter (SEQ ID NO. 9) was amplified using the plasmid pKYLX71:35S2 (Maiti et al., 1993) as a template, with primers designed to permit cloning of the PCR product as a PstI-SalI fragment (CaMV S35 primers: 5′-tttctgcagacaagaagaaaatcttcgtc (SEQ ID NO. 10) and 3′-tttgtcgactttaagcttccttatatagaggaagggtc (SEQ ID NO. 11)). In addition, these primers were designed so that adjacent HindIII and SalI sites would exist at the 3′ end of the fragment. The resulting DNA fragment was cloned into pBluescript as a PstI-SalI fragment. Two copies of the lac operator (SEQ ID NO. 12) were cloned sequentially into this plasmid. First, the plasmid was digested with HindIII and the annealed lacO-HindIII oligonucleotide (SEQ ID NO. 13) ligated with this digested plasmid. The oligonucleotide is designed so that recombinants would lack the HindIII site, allowing enrichment by treating the ligation with HindIII. Similarly, clones containing one copy of the lac operator were digested with SalI and the lacO-SalI oligonucleotide (SEQ ID NO. 14) ligated with this DNA. This plasmid was termed pTD2.

PTD1 was digested with PstI+XhoI and the PstI−XhoI fragment from pTD2 that contained the modified 35S promoter was inserted. The resulting plasmid was digested with XhoI+XbaI and the Sac adapter oligonucleotides (A: tcgagagctct (SEQ ID NO. 15) and B: ctagagagctc (SEQ ID NO. 16) ) inserted. The resulting plasmid was digested with XhoI+SacI and an XhoI—SacI fragment containing the yeast PAB1 gene (Li et al., 2000) (SEQ ID NO. 17) insert. The EcoRI-XbaI fragment from this recombinant was cloned into EcoRI-XbaI-digested pKYLX71:35S2 (Maiti et al., 1993) to yield the final construct shown in FIG. 1. This construct was mobilized into Agrobacterium tumefaciens and the transconjugates were used to transform tobacco as described (e.g., Li et al., 2000).

### EXAMPLE 2

Transgenic plants were made using triparental mating to produce transconjugant Agrobacteria and tobacco transformation as described in Schardl, C. L., Byrd, A. D., Benzion, G., Altshculer, M., Hildebrand, D. F., and Hunt, A. G.(1987) Design and construction of a versatile system for the expression of foreign genes in plants. Gene 61, 1-11.

The constitutive expression of the PR1a gene in kanamycin-resistant R0 transgenic plants that carried the construct shown in FIG. 1 was assessed by Northern blot analysis. Total RNA was isolated from tobacco plants using TRIZOL® Reagent according to the manufacturer's instructions (GIBCO BRL). About 10 μg of total RNA was fractionated in an agarose gel containing 2.2 M formaldehyde, to a membrane and hybridized with a PR1a gene probe that was rabiolabelled using the Prime-It® II Random Primer Labeling Kit (Stratagene). The membrane was dried and exposed to x-ray film. The autoradiogram was visualized using a phosphorimager system. The results are shown in FIG. 2.

The results indicate that about 60% of all tested R0 transformants had a detectable amount of PR1a gene expression. This expression is very unlikely to be due to spurious induction of PR1a gene expression during the process of transformation and regeneration, since analogous screens of other plant lines consistently fail to yield PR1a gene expression (unpublished observations).

### EXAMPLE 3

Several plant lines obtained in Example 1 were selected for further study. Progeny from these lines were tested for resistance to two bacterial pathogens—Erwinia caratovora and Pseudomonas syringae pv. tabaci. The Erwinia tests entailed an accounting of the survival of young seedlings after challenge with the pathogen. Erwinia caratovora subsp. caratovora (from −80° C.) was activated on a LB plate overnight.

A single colony of Erwinia caratovora subsp. caratovora was inoculated into 2 ml of LB medium, cultured at 30° C. overnight, until the onset of stationary phase. The activation in a liquid medium was repeated once, because the process was found to provide a good infection Subsequently, the bacteria were collected by centrifugation at room temperature and suspended in sterile water. The bacterial concentration was monitored at 600 nm and was adjusted to 0.5 (which is approximately 5×10⁸ cells/ml). Two liters of this was used as an inoculum.

Seeds were sterilized by treating with 70% ethyl alcohol (2.5 min), and 5% bleach (15 min). Subsequently the seed was washed with sterile water (one minute) for three times. The sterilized seed was plated on T-Kan (MS salts (4.31 g/l, sugar 30.0 g/l, B5 vitamin stock 2.0 ml/l, and kanamycin 300 mg/l) plate. The seedlings (after 12 days of sowing) were transplanted on 24 well cell culture plates containing T-medium. Inoculation was done after 10 days. For this purpose, minute injury was made on a single leaf of each plant with a pointed forceps, and 2 1 inoculum was deposited on the injured spot. The bacterial drop was allowed to dry by keeping the plants undisturbed in the laminar hood overnight. Subsequently, the plates were incubated in 8 h light and 16 h dark conditions. The plants were monitored for disease symptoms, and the number of dead plants or survived plants was counted after 10-15 days.

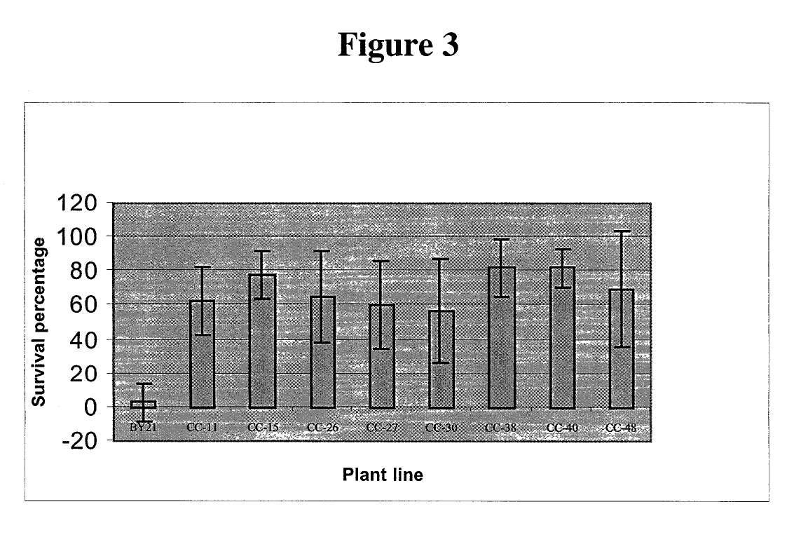

As shown in FIG. 3, fewer than 5% of treated control plants survived the inoculation. In contrast, significant numbers of R1 individuals from 8 independent lines containing the construct survived an identical inoculation (FIG. 3). These numbers ranged from 55 to 80 percent. These results indicate that the progeny of CC R0 plants that possessed elevated PR gene expression also were significantly resistant to the Erwinia pathogen.

Resistance to another pathogen—Pseudomonas syringae pv. tabaci—was also tested. This experiment involved an analysis of the ability of the pathogen to multiply in an infected plant. The bacteria were activated (from −80° C.) by plating on a LB plate and were subsequently grown in King's B medium (protease peptone 10 mg/ml, glycerol 15 mg/ml, K₂HPO₄ 1.5 mg/ml and MgSO₄ 4 mM) overnight at 30° C. The bacteria were collected by centrifugation and resuspended in sterile water. Appropriate dilutions were made so as to obtain OD₆₀₀ of 0.005, 0.01, 0.04 and 0.124 (0.1 OD=10⁸ cells/ml) for inoculation purposes.

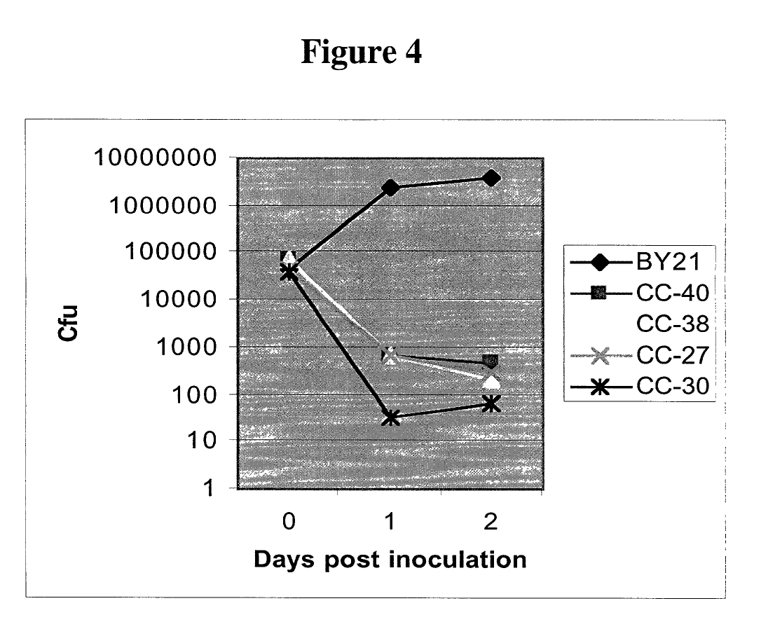

The wt (BY21) and transgenic plants (CC-2#11, 15, 26, 27, 30, 38, 40, and 48) were grown in a green house. The plants used were of 3-4 weeks old (post transplantation). Bacteria at the above mentioned concentrations, and water control was infiltrated on two leaves of each of three repeat plants for wild type (BY21) and the transgenic plants. Before infiltration, a sharp needle was used to make a tiny wound on the leaf panel to facilitate the process. Right after infiltration, two leaf discs were collected from the infiltrated area on each leaf. The leaf discs were rinsed with 70% EtOH for one minute and twice with sterile water. They were ground with ml sterile water. From this serial dilutions (1/10, 1/100, 1/1000, etc) were made from the supernatant, and 100 ml were plated on a LB plate. The plates were incubated at 30° C. for one to two days to count the colonies. The process is repeated for two more days and the observations were recorded. Higher dilutions were made on later days for wild type plants to facilitate the counting of bacterial colonies. The results are shown in FIG. 4.

In wild-type plants, an initial low inoculum multiplied by more than two orders of magnitude over the course of the experiment, as shown in FIG. 4. In contrast, the bacterial pathogen was unable to grow on plants from each of the CC lines that were tested (FIG. 4). Instead, the bacterial population decreased significantly on the inoculated CC plants, indicative of an enhanced defense response against the bacteria.

References

Adam, S. A., Nakagawa, T., Swanson, M. S., Woodruff, T. K., and Dreyfuss, G. (1986). mRNA polyadenylate-binding protein: gene isolation and sequencing and identification of a ribonucleoprotein consensus sequence. Mol Cell Biol 6, 2932-43.

Alfano, J. R., Ki m, H. S., Delaney, T. P., and Collmer, A. (1997). Evidence that the Pseudomonas syringae pv. syringae hrp-linked hrmA gene encodes an Avr-like protein that acts in an hrp-dependent manner within tobacco cells. Mol Plant Microbe Interact 10, 580-8.

An Y Q, McDowell J M, Huang S, McKinney E C, Chambliss S, Meagher R B. (1996) Strong, constitutive expression of the Arabidopsis ACT2/ACT8 actin subclass in vegetative tissues.Plant J. 10,107-21

Bowling, S. A., Clarke, J. D., Liu, Y., Klessig, D. F., and Dong, X. (1997). The cpr5 mutant of Arabidopsis expresses both NPR1-dependent and NPR1-independent resistance. Plant Cell 9, 1573-84.

Brown M, Figge, Hansen U, Wright C, Jeang K T, Khoury G, Livingston D M, Roberts T M. (1987) lac repressor can regulate expression from a hybrid SV40 early promoter containing a lac operator in animal cells. Cell 49, 603-12.

Dangl, J. L., Dietrich, R. A., and Richberg, M. H. (1996). Death don't have no mercy: cell death programs in plant-microbe interactions. Plant Cell 8, 1793-1807.

Foster E, Hattori J, Labbe H, Ouellet T, Fobert P R, James L E, Iyer V N, Miki B L (1999) A tobacco cryptic constitutive promoter, tCUP, revealed by T-DNA tagging. Plant Mol Biol 41, 45-55

Gorlach, J., Volrath, S., Knauf-Beiter, G., Hengy, G., Beckhove, U., Kogel, K. H.,

Oostendorp, M., Staub, T., Ward, E., Kessmann, H., and Ryals, J. (1996). Benzothiadiazole, a novel class of inducers of systemic acquired resistance, activates gene expression and disease resistance in wheat. Plant Cell 8, 629-43.

Hammond-Kosack, K. E., Harrison, K., and Jones, J. D. (1994). Developmentally regulated cell death on expression of the fungal avirulence gene Avr9 in tomato seedlings carrying the disease-resistance gene Cf-9. Proc Natl Acad Sci USA 91, 10445-9.

Hammond-Kosack, K. E., Tang, S., Harrison, K., and Jones, J. D. (1998). The tomato Cf-9 disease resistance gene functions in tobacco and potato to confer responsiveness to the fungal avirulence gene productavr 9. Plant Cell 10, 1251-66.

Li, Q. -S., C. von Lanken, J -J Yang, C. B. Lawrence and A. G. Hunt. (1999). The yeast polyadenylate binding protein (PAB1) gene acts as a disease lesion mimic gene when expressed in plants. Plant Mol. Biol. 42, 335-344.

MacDonald, M. H., Mogen, B., and Hunt, A. G. (1991). Characterization of the polyadenylation signal of the T-DNA-encoded octopine synthase gene. Nucleic Acids Research 19, 5575-5581.

Maiti, I. B., Murphy, J. F., Shaw, J. G., and Hunt, A. G. (1993) Plants that express a potyvirus proteinase gene are resistant to virus infection. Proceedings of the National Academy of Sciences USA 90, 6110-6114.

Morris, S. W., Vernooij, B., Titatam, S., Starrett, M., Thomas, S., Wiltse, C. C., Frederiksen, R. A., Bhandhufalck, A., Hulbert, S., and Uknes, S. (1998). Induced resistance responses in maize. Mol Plant Microbe Interact 11, 643-58.

Oldroyd, G. E. D., and Staskawicz, B. J. (1998). Genetically engineered broad-spectrum disease resistance in tomato. Proc Natl Acad Sci USA 95, 10300-5.

Ohshima M, Matsuoka M, Yamamoto N, Tanaka Y, Kano-Murakami Y, Ozeki Y, Kato A, Harada N, Ohashi Y. (1987) Nucleotide sequence of the PR-1 gene of Nicotiana tabacum. FEBS Lett. 225, 243-246.

Ohshima,M., Harada,N., Matsuoka,M. and Ohashi,Y. (1990) The nucleotide sequence of pathogenesis-related (PR) 1b protein of tobacco. Nucleic Acids Res. 18, 181.

Rao, M. V., and Davis, K. R. (1999). Ozone-induced cell death occurs via two distinct mechanisms in Arabidopsis: the role of salicylic acid. Plant J 17, 603-14.

Ryals, J. A., Neuenschwander, U. H., Willits, M. G., Molina, A., Steiner, H. -Y., and Hunt, M. (1996). Systemic Acquired Resistance. Plant Cell 8, 1809-1819.

Sanger M, Daubert S, Goodman R M. (1990) Characteristics of a strong promoter from figwort mosaic virus: comparison with the analogous 35S promoter from cauliflower mosaic virus and the regulated mannopine synthase promoter. Plant Mol Biol. 14, 433-443.

Shen, S. -H., Q -S. Li, S -Y He and A. G. Hunt. (2000). Broad range pathogen resistance by the expression of a bacterial elicitant in plant. Plant Journal 23, 205-214.

Sticher, L., B. Mauch-Mani, J. P. Metrauz. (1997). Systemic acquired resistance. Ann. Rev. Phytopathol 35, 235-270.

Van Loon, L. C., P. A. H. M. Bakker, and C. M. J. Pieterse (1998). Systemic Resistance Induced By Rhizosphere Bacteria. Annu. Rev. Phytopathol. 36,453-483.

Yu, I. C., Parker, J., and Bent, A. F. (1998). Gene-for-gene disease resistance without the hypersensitive response in Arabidopsis dndl mutant. Proc Natl Acad Sci USA 95, 7819-24.

What is claimed is:

1. An isolated nucleic acid molecule comprising a first polynucleotide encoding an elicitin operably linked to a first plant promoter comprising at least one E. coli lac operator (LacO) located between the promoter TATA box and the translation initiation site of the first polynucleotide, wherein the first plant promoter is constitutive; and a second polynucleotide encoding an E. coli lac repressor (LacI) operably linked to a PR gene promoter.

2. The isolated nucleic acid molecule of claim 1 wherein the first plant promoter comprises two, tandemly aligned E. coli lac operators.

3. The isolated nucleic acid molecule of claim 1 wherein the first plant promoter is a CaMV 35S promoter.

4. The isolated nucleic acid of claim 1 wherein the second plant promoter is a PR-1b promoter.

5. The isolated nucleic acid molecule of claim 1 wherein the first polynucleotide encodes a yeast poly(A) binding protein (Pab1p).

6. The isolated nucleic acid molecule of claim 1 wherein the first polynucleotide encodes a Pseudomonas syringae pv. syringae HrmA gene.

7. An intermediate plant transformation plasmid comprising a region of homology to an Agrobacterium tumefaciens gene vector, an Agrobacterium tumefaciens T-DNA border region and a recombinant nucleic acid construct located between the T-DNA border and the region of homology, wherein the recombinant nucleic acid construct comprises a first polynucleotide encoding an elicitin operably linked to a first plant promoter comprising at least one E. coli lac operator (LacO) located between the promoter TATA box and the translation initiation site of the first polynucleotide, wherein the first plant promoter is constitutive; and a second polynucleotide encoding an E. coli lac repressor (LacI) operably linked to a PR gene promoter.

8. The intermediate plant transformation plasmid of claim 7 wherein the first plant promoter of the construct comprises two, tandemly aligned E. coli lac operators.

9. The intermediate plant transformation plasmid of claim 8 wherein the first plant promoter is a CaMV 35S promoter.

10. The intermediate plant transformation plasmid of claim 7 wherein the PR gene promoter is a PR-1b promoter.

11. The intermediate plant transformation plasmid of claim 7 wherein the first polynucleotide encodes a yeast poly(A) binding protein (Pab-1p).

12. The intermediate plant transformation plasmid of claim 7 wherein the first polynucleotide encodes a Pseudomonas syringae pv. syringae HrmA gene.

13. A plant transformation vector comprising a disarmed Agrobacterium tumefaciens plant tumor-inducing plasmid and a recombinant nucleic acid construct comprising a first polynucleotide encoding an elicitin operably linked to a first plant promoter comprising at least one E. coli lac operator (LacO) located between the promoter TATA box and the translation initiation site of the first polynucleotide, wherein the first plant promoter is constitutive; and a second polynucleotide encoding an E. coli lac repressor (LacI) operably linked to a PR gene promoter.

14. A plant transformation vector of claim 13 wherein first plant promoter of the construct comprises two, tandemly aligned E. coli lac operators.

15. The plant transformation vector of claim 14 wherein the first plant promoter is a CaMV 35S promoter.

16. The plant transformation vector of claim 13 wherein the PR gene promoter is a PR-1b promoter.

17. The plant transformation vector of claim 13 wherein the first polynucleotide encodes a yeast poly(A) binding protein (Pab1p).

18. The plant transformation vector of claim 13 wherein the first polynucleotide encodes a Pseudomonas syringae pv. syringae HrmA gene.

19. A transformed plant protoplast comprising a plant transformation vector comprising a disarmed Agrobacterium tumefaciens plant tumor-inducing plasmid and a recombinant nucleotide construct comprising a first polynucleotide encoding an elicitin operably linked to a first plant promoter comprising at least one E. coil lac operator (LacO) located between the promoter TATA box and the translation initiation site of the first polynucleotide, wherein the first plant promoter is constitutive; and a second polynucleotide encoding an E. coli lac repressor (LacI) operably linked to a PR gene promoter.

20. The transformed plant protoplast of claim 19 comprising two, tandemly aligned E. coil lac operators.

21. The transformed plant protoplast of claim 20 wherein the first plant promoter is a CaMV 35S promoter.

22. The transformed plant protoplast of claim 19 wherein the PR gene promoter is a PR-1b promoter.

23. The transformed plant protoplast of claim 19 wherein the first polynucleotide encodes a yeast poly(A) binding protein (Pab1p).

24. The transformed plant protoplast of claim 19 wherein the first polynucleotide encodes a Pseudomonas syringae pv. syringae HrmA gene.

25. A method of producing a transgenic plant with increased disease resistance comprising:
1) providing a nucleic acid construct comprising a first polynucleotide encoding an elicitin operably linked to a first plant promoter comprising at least one E. coli lac operator (LacO) located between the promoter TATA box and the translation initiation site of the first polynucleotide, wherein the first plant promoter is constitutive; and a second polynucleotide encoding an E. coli lac repressor (LacI) operably linked to a PR gene promoter;
2) introducing the construct into a plant tissue to produce transgenic plant tissue; and
3) regenerating the transgenic plant tissue to produce a transgenic plant, whereby the elicitin is expressed, thereby inducing a hypersensitive and/or SAR response, which induces lac repressor expression, which regulates the expression of the elicin.

26. The method of claim 25 wherein the first promoter comprises two, tandemly aligned E. coli lac operators.

27. The method of claim 26 wherein the first plant promoter is a CaMV 35S promoter.

28. The method of claim 25 wherein the PR gene promoter is a PR-1b promoter.

29. The method of claim 25 wherein the first polynucleotide encodes a yeast poly(A) binding protein (Pab1p).

30. The method of claim 25 wherein the first polynucleotide encodes a Pseudomonas syringae pv. syringae HrmA gene.

31. A transgenic plant comprising a nucleic acid construct comprising a first polynucleotide encoding an elicitin operably linked to a first plant promoter comprising at least one E. coli lac operator (LacO) located between the promoter TATA box and the translation initiation site of the first polynucleotide, wherein the first plant promoter is constitutive; and a second polynucleotide encoding an E. coli lac repressor (LacI) operably linked to a PR gene.

32. The transgenic plant of claim 31 wherein the first promoter comprises two, tandemly aligned E. coli lac operators.

33. The transgenic plant of claim 32 wherein the first plant promoter is a CaMV 35S promoter.

34. The transgenic plant of claim 31 wherein the PR gene promoter is a PR-1b promoter.

35. The transgenic plant of claim 31 wherein the first polynucleotide encodes a yeast poly(A) binding protein (Pab1p).

36. The transgenic plant of claim 31 wherein the first polynucleotide encodes a Pseudomonas syringae pv. syringae HrmA gene.
